(19) United States
(12) Patent Application Publication
(10) Pub. No.: US 2006/0052722 A1
(43) Pub. Date: Mar. 9, 2006

(54) Ultra-fine micropsy needle

(76) Inventors: Robert T. Brautigam, Glastonbury, CT (US); George A. Perdrizet, Glastonbury, CT (US); Richard H. Brautigam, Newark, DE (US)

Correspondence Address: Connolly Bove Lodge & Hutz, LLP, P.O. Box 2207, Wilmington, DE, 19899-2207, US

(21) Appl. No.: 10/937,118

(22) Filed: Sep. 9, 2004

(51) Int. Cl. A61B 10/00; A61B 17/32

(52) U.S. Cl. 600/567; 606/180; 606/167

(57) ABSTRACT

A specimen retrieving needle is in the form of an elongated body having a closed lead end. At least one cutting blade may be formed on the body. The blade may have a sharpened edge so as to remove a specimen from an internal organ. The needle is ultrathin with an outside diameter of less than 1.0 mm to minimize tissue trauma and to reduce the risk of pain, hemorrhage and organ damage.

### BACKGROUND OF THE INVENTION

Conventional biopsy needles and devices are designed to retrieve a sample or specimen that has an adequate size required for conventional histologic, cytologic or protein analyses. The actual size of current devices is ultimately dictated by the tissue and disease process that is the center of investigation. Generally, the size is not less than 14-16 Ga (0.065-0.083 inches OD or 0.047-0.063 inches ID). Smaller tissue samples can be obtained through a process of fine-needle aspiration. This technique employs 18-2 OGa needles, but samples are inadequate for conventional histologic analysis and provide only cytological information. Currently the major limitations to these biopsy techniques are the associated tissue injury and associated complications. Complications include pain during the procedure, bleeding, organ damage and subsequent infection. Clinicians constantly must weigh the risk of the biopsy procedure with potential benefits to the patient. The risks of complication from a technical-device standpoint are directly related to the diameter of the biopsy needle. Thus there are many situations in which tissues are not biopsied because of an unfavorable risk/benefit ratio.

An ultra-fine biopsy needle would dramatically alter the risk/benefit ratio when it comes to the percutaneous sampling of human tissues for diagnostic purposes. Until very recently there has not been a need to have a needle smaller than the 20 Ga instrument used for fine-needle aspirations (although there certainly has been a desire/wish on the part of the patient and physician). The recent arrival of several new and synergistic technologies has changed all of this. The new technologies are the DNA and RNA microarray methodologies (so-called “gene chips”), proteomics and computer based software for analysis (bioinformatics) of the large volumes of data generated by these techniques (a.k.a. molecular-genetic techniques). Superimposed on these methodologies is the recent completion of the sequencing of the human genome which will allow meaningful interpretation of the molecular biologic data. These two synergistic technologies are in their infancy but rapidly evolving and will likely revolutionize the practice of medicine. To realize the full potential of these technologies, it is necessary that DNA/RNA and protein samples be easily and safely obtained from the tissue of interest. The tremendous power of these techniques allows extensive studies to be preformed on minute tissue samples. Conventional techniques (standard histologic, cytologic and protein gel electrophoresis) require tissues quantities that range from the milligram-to-gram size while the new technologies permit the use of nanogram-to-microgram quantities. The quantity of tissue required by these techniques is astonishingly small when compared to today's standards. Thus with less tissue we can now retrieve much more information that was ever thought possible! Currently, with this early microarray technology, it is possible to simultaneously study over 1200 genes using a single sample having a size of only 1-50 micrograms. The medical literature is now reporting the first human studies, which have employed the new genetic technology.

We anticipate a very large demand for obtaining tissues that are of adequate dimensions to provide material to perform modern molecular genetic assays. Because the acceptable sample size is so small, the dimensions of the biopsy needle can now be reduced to the limits of physical engineering. Ideally a needle could be made so small that it may be painless and completely safe, while still able to provide the necessary sample size employed by these assays. The enabling technologies associated with the molecular-genetic revolution will only grow with time and so too will the demand for human tissue samples. During the 2000 meeting of the American Chemical Society, one of the hottest topics of discussion were DNA microarray systems. The National Institute of Health has established a National Human Genome Research Institute which clearly demonstrates the dimensions of this new molecular genetic technology. Further, the successful sequencing of the human genome is only the beginning of the application of molecular genetics to human diseases. Currently there are approximately 30,000 genes that reside within the human genome, of these the vast majority are of unknown function and importance. The medical sciences are now embarking on a systematic study of these genes. As this understanding grows, so too will the medical applications of this new knowledge. The one process standing between these technologies and their application to everyday medical care will be the safe, accurate and successful retrieval of human tissue to study. Current biopsy needles could provide these methods with adequate tissue, without a doubt. However, by realizing that minute amounts of tissue can be used, then the novel idea that an ultra-fine biopsy needle would enable tremendous freedom to biopsy tissues in clinical situations which would be prohibitive with conventional devices becomes an important insight into the state of the art of biopsy technology. Finally, all of what has been said regarding the needle in the setting of human subjects will also hold true for many animal models used in medical scientific investigations. The need is great and the potential market is tremendous.

### SUMMARY OF THE INVENTION

An object of this invention is to provide an ultra-fine micropsy needle which satisfies the above needs.

A further object of this invention is to provide such an ultra-fine needle which can be easily manipulated for retrieving a specimen.

A goal of this invention is the development of an ultra-fine biopsy needle that: 1) may penetrate the skin between nerve endings and therefore be painless, and 2) may inflict minimal tissue trauma because of the small diameter and small size of the sample removed, thus dramatically reducing the risk of hemorrhage and organ damage as an unwanted side effect of all biopsy techniques. If such a needle were to exist then patients would readily accept the procedure, physicians would readily prescribe the procedure and both would benefit from the tremendous strength of the new genetic technologies. The development of the “micropsy” needle would provide an enabling technology that will become a fundamental component in the future practice of molecular medicine.

In accordance with this invention the ultra-fine micropsy needle has an outside diameter of less than 1.0 mm and preferably in the range of 0.01 mm to less than 1.0 mm. The maximum outside diameter could be no greater than 0.8 mm or no greater than 0.6 mm or no greater than 0.4 mm or no greater than 0.2 mm or no greater than 0.1 mm.

The invention could be practiced where the needle is a specimen retrieving needle having structure for cutting and then retrieving a specimen.

In accordance with one practice of this invention the specimen retrieving needle has a closed lead end to facilitate its movement into the body without collecting tissue or other matter. At least one blade or specimen retrieving member is formed in the wall of the needle. The needle also includes structure to collect or hold the specimen while the needle is removed from the body. The blade is bent at a non-radial or non-circumferential direction with respect to the longitudinal axis of the needle to form a rigid flap with its angle of extension minimized and so that the needle can be inserted in a rotating motion opposite to the direction of the sharp edge of the blade.

In one practice of the invention a pair of diametrically opposite blades is provided for a hollow needle. One blade would extend at an angle to provide for a digging action while the other would be more inclined and would provide for controlled slicing. The interior of the needle would be exposed at the location of each blade to permit the specimen to thereby enter the needle at that location for retrieval purposes.

### THE DRAWINGS

FIG. 1 is a schematic view of a specimen retrieving needle in accordance with this invention;

FIG. 2 is a side elevation partly in section of a portion of the needle shown in FIG. 1;

FIG. 3 is a cross-sectional view taken through FIG. 2 along the line 3-3;

FIG. 4 is a view similar to FIG. 3 showing modified forms of the cutting blades;

FIGS. 5A, 5B and 5C are schematic cross-sectional views showing different stages of use of the needle in accordance with this invention; and

FIGS. 6 and 7 are side elevational views showing alternative tip formations for a specimen retrieving needle in accordance with this invention.

### DETAILED DESCRIPTION

The present invention is directed to providing an ultrafine micropsy needle which would dramatically alter the risk/benefit ratio when it comes to the percutaneous sampling of human tissues for diagnostic purposes. In accordance with this invention the ultra-fine micropsy needle has an outside diameter of less than 1.0 mm and preferably in the range of 0.01 mm to less than 1.0 mm. The maximum outside diameter could be no greater than 0.8 mm or no greater than 0.6 mm or no greater than 0.4 mm or no greater than 0.2 mm or no greater than 0.1 mm.

In order to provide such a needle proper manufacturing techniques and materials would have to be used to attain the functional designs of the desired dimensions.

The invention could be practiced where the needle is a specimen retrieving needle having structure for cutting and then retrieving a specimen.

In accordance with this invention a specimen retrieving needle is provided which is may be considered as an ultra-fine micropsy needle or an ultrathin biopsy needle wherein the lead end of the instrument or needle is closed so that the needle will not pick up or collect or otherwise be filled with tissue or other matter while the needle is being inserted to its desired location. The needle may include structure for removing a specimen and structure for collecting or holding the specimen when the needle is being withdrawn from the body. The invention may be practiced with various types of needles and various types of removal/collecting structure and with the components of the invention made of various materials and sizes. For example, where the needle is ultrathin the lead end could be blunt or rounded, but not pointed so as to avoid the possibility of piercing a nerve ending. Instead, the needle would penetrate the skin between nerve endings and therefore be painless. If desired, however, the needle could have a pointed or tapered lead end to facilitate its insertion into the body. The needle itself could be hollow or could be solid. Where a hollow needle is provided the hollow interior could serve as the collection structure for retaining a specimen during needle removal. Where the needle is solid, structure could be provided on the outer surface or in a recess in the outer surface of the needle to function as the collecting structure for the specimen. Preferably, the needle will be small enough in diameter to cause minimal nervous stimulation. That is, i.e, to enable the needle to fit between nerve endings. Preferably, the needle is circular in cross-section, although other shapes could be used within the broad practice of this invention such as oval, octagonal, etc.

Based upon the foregoing, the invention will now be described with respect to some possible structural forms for the needle. It should be understood, however, that these structural forms are merely exemplary practices of the invention and are not intended to limit how the invention would be practiced.

As shown in FIGS. 1-2 the specimen retrieving needle 10 is generally of cylindrical shape having a hollow needle body 12. The lead end of needle 10 has a pointed conical tip 14 to facilitate the insertion of the needle into the body of the patient so that the needle could ultimately be inserted into an internal organ 16 in a known manner, such as by mounting on a suitable inserting mechanism. One example of an inserting mechanism is a guide wire which might be 4-5 inches long. Tip 14 is preferably permanently secured to body 12 in any suitable manner such as by welding. As shown in FIG. 2 the tip 14 includes a recessed base 18 which fits snugly in the interior of body 12 and then is welded in place. Alternatively, the tip 14 could be formed from the needle body.

In accordance with a practice of this invention at least one cutting blade is provided on the needle body to function as specimen removal structure. FIGS. 1-5 illustrate a practice of the invention wherein two circumferentially spaced cutting blades 20,22 are provided preferably diametrically opposite each other. Each blade may be formed by cutting a section of body 12 and bending the section outward so as to form a rigid flap and create an opening 24 at the location of each blade. FIG. 2, for example, shows opening 24 located at blade 22. Each blade may have a sharpened remote longitudinal edge 26 which could be serrated as shown in FIG. 2 or could be a straight edge. If desired one blade 20 may have a straight edge while the other blade 22 may have the serrated edge or both edges could be the same. If desired a transverse side 27 of each blade or flap or a transverse edge 29 of the body of the needle may be sharpened.

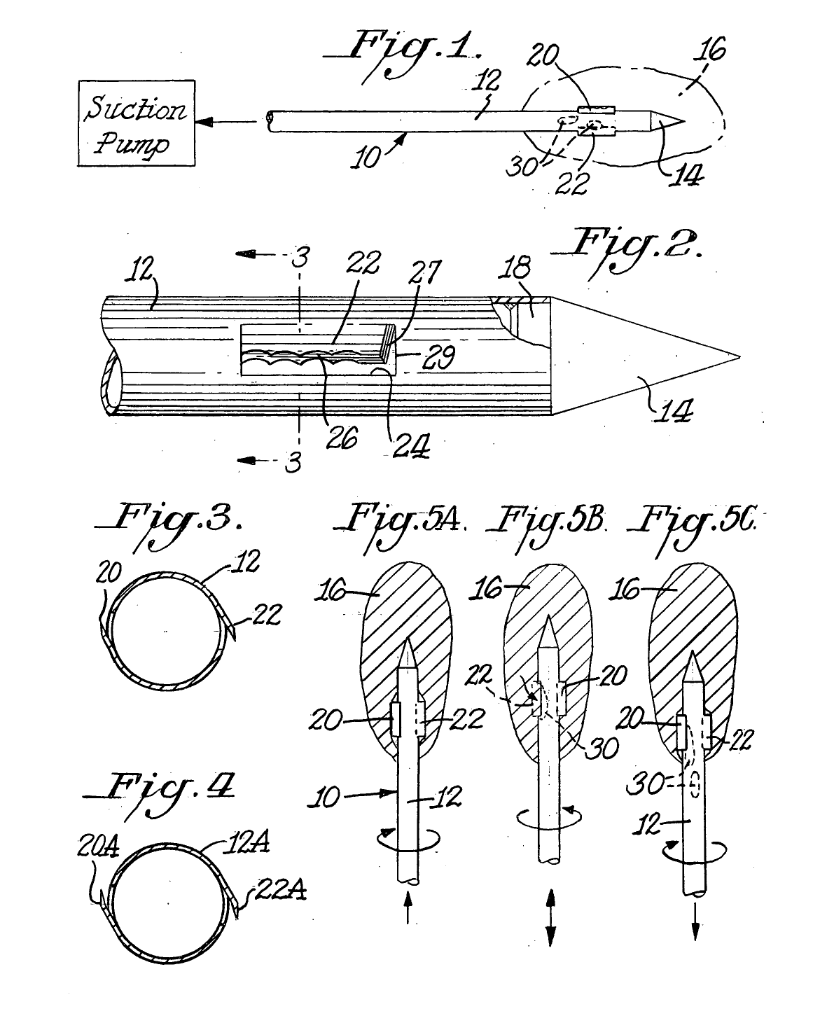

The blades could be formed at an angle so that either a digging action is achieved or a controlled slicing action is achieved. FIG. 3, for example, shows the blades 20, 22 to extend in a direction completely tangential to body 12 to achieve a digging action. FIG. 4, however, shows the ends of blades 20A, 22A to be bent at a shallower angle closer or less than tangential to the outer surface of body 12 for providing a controlled slice thickness cut. Thus, the controlled thickness blades are flatter and extend less outwardly than the digging type blades of FIG. 3.

A difference between the blades which perform a digging action as compared to the blades which have controlled slicing is that the controlled slicing blades after extending away from the body 12 are then disposed in an orientation which is closer to the body than is the more sharply angled digging blades.

The invention may be practiced with any suitable number of blades or specimen removal structure located on the needle. Thus, while at least one blade or set of structures should be provided and two blades are illustrated along the side of the needle, any number, including more than two could be used if appropriate. Similarly, the location of the blades or removal structure could be varied so as to be along the side of the needle proximal the lead end or even at the lead end itself or at some location distal from the lead end. It is expected that where the needle is ultrathin the needle will be so small that it will become plugged or filled once deployed and thus the cutting edges or blades will no longer cut. The particular type of tissue removal could also vary such as sawing, chopping or rotating.

If desired, the needle could incorporate both blades being of the same type, either digging or controlled slicing or one blade could be digging and the other controlled slicing or only one blade could be provided rather than two blades.

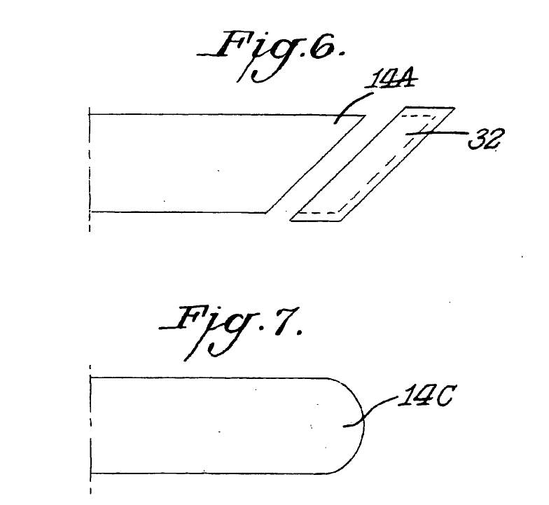

FIG. 5A shows the needle 10 during its insertion movement. As shown therein needle 10 would be rotated in a clockwise direction so that the blades 20, 22 do not cut into the patient during the insertion movement while the needle is being placed into the desired location in the organ 16. When the desired location has been reached needle 10 is rotated counterclockwise as shown in FIG. 5B to cut or slice the specimen from the organ. The cut specimen is also located at the opening 24 in a position to enter the hollow interior of needle body 12. Any suitable means, such as suction, could be employed to then pull the specimen into the hollow body 12.

Preferably suction is not used. Instead, the specimen simply remains in the hollow interior of the needle. The use of a sharp edge, such as edge 27 on blade 22 or 20 or a sharp edge 29 on the needle body 12 might be desirable to further facilitate the cutting of the specimen from the organ. If desired, the cutting or digging action could also be facilitated by a slight in and out reciprocal motion along the axis of the needle 12 to facilitate the edge 26 completely cutting the specimen.

After the specimen 30 is free and has entered the hollow interior of body 12 the needle 10 is then removed by again rotating the clockwise direction shown in FIG. 5C while being pulled outwardly from the organ 16.

The invention may also be practiced where instead of forming the blades as rigid flaps in the body 12 the blades are separate members secured to the exterior of the body. Where the blades are separate members they are preferably permanently secured to the body. If desired, the blades can be removably secured. Where the blades are formed in that manner, as separate members, it is not necessary that the needle be hollow since the blades would function as the retrieving member.

If desired, the specimen need not be retrieved by being pulled into the interior of body 12, but may remain on the outer surface of the blade whereby the surface of the blade and/or the outer surface of the needle would function as the specimen collecting structure particularly where a solid needle is used.

Although FIGS. 1, 2 and 5 illustrate the needle to have a pointed tip, the invention may be practiced with other types of tip structure. FIG. 6, for example, illustrates a tapered tip 14A. If desired a cap 32 could be provided to protect the edge of tapered tip 14A so that the tip is shielded until the time of use. Such a protective cap could also be provided for the pointed end 14 of FIGS. 1, 2 and 5.

Although specific practices of the invention have been described wherein the needle has a pointed tip, such a pointed tip may not be necessary particularly where a very small diameter needle is used. In such case a rounded tip such as tip 14C of FIG. 7 or even a blunt tip could be provided at the lead end of the hollow or solid needle. As a result, the danger of piercing nerves or tissue is minimized and the needle is inserted by passing between nerve endings. Where the needle is in the form of an ultra-fine biopsy needle of very small size, minimal tissue trauma would be inflicted because of the small diameter of the needle and because of the small size of the sample or specimen being removed. This would dramatically reduce the hemorrhage and organ damage as an unwanted side effect of all biopsy techniques. Thus, the patients would readily accept the procedure and physicians would readily prescribe the procedure with both patients and physicians thereby benefitting from the tremendous strength of the new genetic technologies.

What is claimed is:

1. An ultra-fine micropsy needle comprising an elongated body, and said body having an outside diameter of less than 1.0 mm.

2. The needle of claim 1 wherein said body has a closed lead end, removal structure on said body for removing a specimen from an organ or the like, and collecting structure on said body for holding the removed specimen during manipulation of said needle while the needle is being withdrawn from a patient.

3. The needle of claim 2 wherein said removal structure comprises at least one blade extending outwardly from said body.

4. The needle of claim 3 wherein said body has a hollow interior, said blade comprising a rigid flap bent outwardly from said body to expose said hollow interior of said body, and said hollow interior being said collecting structure.

5. The needle of claim 4 wherein said blade has a sharpened longitudinal edge.

6. The needle of claim 5 wherein said blade has a sharpened transverse edge.

7. The needle of claim 4 wherein said blade is at an angle tangential to said body.

8. The needle of claim 4 wherein said blade is at an angle which is less than tangential to said body.

9. The needle of claim 2 wherein said removal structure comprises at least two circumferentially spaced blades extending outwardly from said body.

10. The needle of claim 9 wherein said body has a hollow interior, each of said blades comprising a rigid flap bent outwardly from said body to expose said hollow interior, and said hollow interior being said collecting structure.

11. The needle of claim 10 wherein at least one of said blades has a sharpened longitudinal edge.

12. The needle of claim 11 wherein at least one of said blades has a sharpened transverse edge.

13. The needle of claim 10 wherein each of said blades has a sharpened longitudinal edge.

14. The needle of claim 10 wherein at least one of said blades is at an angle tangential to said body.

15. The needle of claim 10 wherein at least one of said blades is at an angle less than tangential to said body.

16. The needle of claim 10 wherein one of said blades is at an angle tangential to said body, and another one of said blades is at an angle less than tangential to said body.

17. The needle of claim 16 wherein said at least two blades comprises two diametrically spaced blades.

18. The needle of claim 2 wherein said lead end in pointed.

19. The needle of claim 2 wherein said lead end is non-pointed.

20. The needle of claim 2 wherein said body is solid, and said collecting structure being on the outer surface of said body.

21. The needle of claim 1 wherein said body has an outside diameter in the range of 0.01 mm to no greater than 0.8 mm.

22. The method of retrieving a specimen through the use of an ultra-fine needle having an outside diameter of less than 1.0 mm, comprising inserting the needle into the patient while rotating the needle in a first direction, continuing the insertion until the needle is in the desired location, reversing the direction of rotation of the needle to cut or slice the specimen from an organ by at least one blade extending outwardly from the body of the needle, collecting the specimen in the hollow interior of the body of the needle, and withdrawing the needle from the patient while rotating the needle in the first direction.
